(19) United States
(12) Patent Application Publication
(10) Pub. No.: US 2025/0157637 A1
(43) Pub. Date: May 15, 2025

(54) HEALTHCARE COORDINATION PLATFORM AND USER INTERFACE

(71) Applicants: Ashley Patrice BARROW, Cincinnati, OH (US); Aaron Mauric BESS, JR., Cincinnati, OH (US)

(72) Inventors: Ashley Patrice BARROW, Cincinnati, OH (US); Aaron Mauric BESS, JR., Cincinnati, OH (US)

(21) Appl. No.: 18/944,481

(22) Filed: Nov. 12, 2024

Related U.S. Application Data
(60) Provisional application No. 63/598,140, filed on Nov. 13, 2023.

(51) Int. Cl. G16H 40/20; G16H 70/60; G16H 80/00

(52) U.S. Cl. CPC G16H 40/20; G16H 70/60; G16H 80/00

(57) ABSTRACT

A computer-implemented method is provided, comprising: receiving electronic health data associated with a patient; identifying, by a first machine learning model, one or more medical condition keywords from the electronic health data associated with the patient; determining, by a second machine learning model, one or more recommended care providers for the patient based on the identified one or more medical condition keywords; generating, using the second machine learning model, an ordered list of the one or more recommended care providers; and displaying the ordered list of the one or more recommended care providers on a user interface.

### CROSS-REFERENCE TO RELATED APPLICATIONS

This application claims the benefit of U.S. Provisional Application No. 63/598,140, filed Nov. 13, 2023, the entire contents of which are hereby incorporated by reference herein.

### FIELD

This disclosure relates generally to healthcare management, and more specifically, to systems and methods of coordinating patient care using artificial intelligence.

### BACKGROUND

The healthcare system faces significant challenges due to fragmented care coordination and inefficient processes for care plan generation and claims management. Care coordinators, healthcare providers, and hospitals often struggle to create effective care plans that are personalized to the needs of the patient, resulting in suboptimal patient outcomes and elevated readmission rates. Further, care plans are often provided to patients in a disorganized and overwhelming format, making it more difficult for patients to make informed decisions regarding their care and to understand what actions are needed for compliance with their care provider's recommendations.

### SUMMARY

Accordingly, it would be beneficial to have the ability to create personalized care plans that account for the individualized needs of the patient while providing information in an organized format that the patient can easily understand. Provided herein are systems, methods, and user interfaces that can leverage artificial intelligence to create personalized care plans that can lead to improved care outcomes and patient experiences. For example, a machine learning model may be used to generate a personalized care plan based on electronic health data associated with a patient. A machine learning model may be trained to recognize medical keywords in de-identified electronic health records (EHR), and a machine learning model may use these keywords, along with learned information from actual care providers, to locate care provider referrals that suit the patient's needs. The machine learning model may also be trained to recognize social determinants of health (SDoH) in patient data and may recommend supportive services (e.g. housing assistance or food security resources) to improve patient outcomes for patients of all socioeconomic backgrounds. Additionally, a machine learning model may be trained to predict a patient's readmission risk, which may inform a care provider's decisions and allow care providers to take preventative measures to reduce the chance of readmission.

The care providers recommended by a machine learning model may be displayed in a user interface that may be accessible to both the patient and to a care provider. Through the user interface, a care provider may filter and edit the recommended care providers based on primary and secondary care needs of the patient, location, and/or insurance providers accepted. A patient may also be able to view their care plan, access educational and health literacy information, and perform tasks such as scheduling appointments and ordering prescription refills. The user interface may include a feature for consolidating the list of recommended care providers into an integrated care plan, which may include contact information for each care provider as well as descriptions of services offered and explanations behind each recommendation in an easy-to-understand format.

The integrated care plan may be displayed and/or printed out and provided to a patient so that they have all the information they need to make informed decisions about their care in one document, as opposed to the disorganized piles of paperwork that are typically administered at patient discharge. These functionalities may be supported by a microservices-based system that ensures seamless interaction between artificial intelligence services and external healthcare systems and insurance systems. The systems, methods, and user interfaces provided can thus help to improve access to care, care plan compliance, and overall patient outcomes and experiences, thereby reducing the amount of patient readmissions and easing the existing strain on healthcare systems.

In some examples, a computer-implemented method is provided, comprising: receiving electronic health data associated with a patient; identifying, and identifying by a first machine learning model, one or more medical condition keywords from the electronic health data associated with the patient. The first machine learning model may be trained to identify medical condition keywords from training data comprising de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients. The computer-implemented method may include determining, by a second machine learning model, one or more recommended care providers for the patient based on the identified one or more medical condition keywords; and generating, using the second machine learning model, an ordered list of the one or more recommended care providers. The second machine learning model may be trained to generate the ordered list from training data comprising medical condition training keywords paired with recommendations by care providers of other care providers treating one or more of the medical conditions indicated by the training keywords. The computer-implemented method may include displaying the ordered list of the one or more recommended care providers on a user interface.

In some examples, the computer-implemented method includes displaying an interactive map on the user interface indicating locations of the one or more recommended care providers.

In some examples, the computer-implemented method includes determining a first set of filters for filtering the ordered list by one or more primary services, and a second set of filters for filtering the ordered list by one or more secondary services; and displaying a first dropdown menu with the first set of filters and a second dropdown menu with the second set of filters on the user interface.

In some examples, the computer-implemented method includes receiving, at the user interface, a user selection comprising one or more primary services from the first dropdown menu, one or more secondary services from the second dropdown menu, or both; and updating the ordered list of the one or more recommended care providers based on the user selection.

In some examples, the one or more primary services, the one or more secondary services, or both comprise literacy support services, housing support services, transportation support services, food security resources, financial support resources, or any combination thereof.

In some examples, the computer-implemented method includes determining, by a third machine learning model, a readmission risk for the patient based on the electronic health data. The third machine learning model may be trained to determine a readmission risk from training data comprising de-identified electronic health records paired with one or more readmission-causing events. The method may include displaying an indication of the readmission risk for the patient on the user interface.

In some examples, the computer-implemented method includes identifying, by a fourth machine learning model, one or more current procedural technology (CPT) codes in the electronic health data associated with the patient. The fourth machine learning model may be trained to identify the one or more CPT codes from training data comprising de-identified health records paired with corresponding CPT codes. The method may include generating, by the third machine learning model, an insurance claim based on the one or more CPT codes.

In some examples, the computer-implemented method includes receiving, at the user interface, a user selection of a visual affordance for generating an integrated care plan; in response to receiving the user selection of the visual affordance, and generating, using the second machine learning model, an integrated care plan for the patient. The integrated care plan may include the ordered list of the one or more recommended care providers and a natural language explanation for why each of the one or more recommended care providers were recommended. The integrated care plan may be displayed on the user interface.

In some examples, the integrated care plan includes, for each of the one or more recommended care providers, a care provider location, services offered, insurance providers accepted, cost information, or any combination thereof.

In some examples, the computer-implemented method includes. prior to receiving electronic health data associated with a patient: receiving unstructured electronic health information associated with the patient; and converting, using a fast healthcare interoperability resources application programming interface (FHIR API), the unstructured electronic health information into electronic health data associated with the patient, the electronic health data comprising a FHIR data format.

In some examples, a system is provided that includes one or more processors and memory. The memory may store computer program code executable by the one or more processors to cause the system to: receive electronic health data associated with a patient; and identify, by a first machine learning model, one or more medical condition keywords from the electronic health data associated with the patient. The first machine learning model may be trained from training data that includes de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients. The system may be caused to determine, by a second machine learning model, one or more recommended care providers for the patient based on the identified one or more medical condition keywords; and generate, using the second machine learning model, an ordered list of the one or more recommended care providers. The second machine learning model may be trained from training data including medical condition training keywords paired with recommendations by care providers of other care providers treating one or more of the medical conditions indicated by the training keywords. The system may be caused to display the ordered list of the one or more recommended care providers on a user interface.

In some examples, the system is caused to display an interactive map on the user interface indicating locations of the one or more recommended care providers.

In some examples, the system is caused to determine a first set of filters for filtering the ordered list by one or more primary services, and a second set of filters for filtering the ordered list by one or more secondary services; and display a first dropdown menu with the first set of filters and a second dropdown menu with the second set of filters on the user interface.

In some examples, the system is caused to receive, at the user interface, a user selection comprising one or more primary services from the first dropdown menu, one or more secondary services from the second dropdown menu, or both; and update the ordered list of the one or more recommended care providers based on the user selection.

In some examples, the one or more primary services, the one or more secondary services, or both comprise literacy support services, housing support services, transportation support services, food security resources, financial support resources, or any combination thereof.

In some examples, the system is caused to determine, by a third machine learning model, a readmission risk for the patient based on the electronic health data. The third machine learning model may be trained from training data comprising de-identified electronic health records paired with one or more readmission-causing events. The system may be caused to display an indication of the readmission risk for the patient on the user interface.

In some examples, the system is caused to identify, by a fourth machine learning model, one or more current procedural technology (CPT) codes in the electronic health data associated with the patient. The fourth machine learning model may be trained to identify the one or more CPT codes from training data comprising de-identified health records paired with corresponding CPT codes. The system may be caused to generate, by the fourth machine learning model, an insurance claim based on the one or more CPT codes.

In some examples, the system is caused to receive, at the user interface, a user selection of a visual affordance for generating an integrated care plan; and in response to receiving the user selection of the visual affordance, generate, using the second machine learning model, an integrated care plan for the patient. The integrated care plan may include the ordered list of the one or more recommended care providers and a natural language explanation for why each of the one or more recommended care providers were recommended. The system may be caused to display the integrated care plan on the user interface.

In some examples, the system is caused to, prior to receiving electronic health data associated with a patient: receive unstructured electronic health information associated with a patient; and convert, using a fast healthcare interoperability resources application programming interface (FHIR API), the unstructured electronic health information into electronic health data associated with the patient, the electronic health data comprising a FHIR data format.

In some examples, a non-transitory computer readable storage medium storing one or more programs is provided. The one or more programs may include instructions, which, when executed by a system comprising one or more processors and memory, cause the system to: receive electronic health data associated with a patient; and identify, by a first machine learning model, one or more medical condition keywords from the electronic health data associated with the patient. The first machine learning model may be trained to identify medical condition keywords from training data comprising de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients. The system may be caused to determine, by a second machine learning model, one or more recommended care providers for the patient based on the identified one or more medical condition keywords; and generate, using the second machine learning model, an ordered list of the one or more recommended care providers. The second machine learning model may be trained to generate the ordered list from training data comprising medical condition training keywords paired with recommendations by care providers of other care providers treating one or more of the medical conditions indicated by the training keywords. The system may be caused to display the ordered list of the one or more recommended care providers on a user interface.

In some embodiments, any one or more of the characteristics of any one or more of the systems, methods, and/or computer-readable storage mediums recited above may be combined, in whole or in part, with one another and/or with any other features or characteristics described elsewhere herein.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary care coordination system, according to some embodiments.

FIG. 2 illustrates an exemplary computer-implemented method, according to some embodiments.

FIG. 3 illustrates a patient overview page in an exemplary user interface, according to some embodiments.

FIG. 4 illustrates an exemplary view of a list of recommended care providers in a user interface, according to some embodiments.

FIG. 5 illustrates exemplary dropdown menus for filtering a list of recommended care providers, according to some embodiments.

FIG. 6 illustrates an exemplary care provider page, according to some embodiments.

FIG. 7 illustrates an exemplary print view of an integrated care plan, according to some embodiments.

FIG. 8 illustrates an exemplary print view of an integrated care plan, according to some embodiments.

FIG. 9 illustrates an exemplary provider profile in a user interface, according to some embodiments.

FIG. 10 illustrates an exemplary account creation menu, according to some embodiments.

FIG. 11 illustrates an exemplary account creation alert, according to some embodiments.

FIG. 12 illustrates a computer, according to some embodiments.

### DETAILED DESCRIPTION

Provided are systems, methods, and user interfaces that may improve patient care, reduce patient readmissions, and facilitate seamless communication between patients and their care providers. In some examples, a method of generating a personalized care plan may utilize one or more machine learning models that can securely access and retrieve electronic health data associated with the patient and identify medical condition keywords in the electronic health data. A machine learning model may be trained to perform this function by training inputs comprising de-identified sets of EHR paired with medical condition keywords associated with the de-identified patient as outputs. A second machine learning model may use these keywords to match the patient to care providers that offer services tailored to treat the identified medical conditions. A care provider may utilize a user interface to edit the list of recommended care providers, and an integrated care plan for the patient may be displayed on user interface that includes all the information the patient could need to easily identify the next steps in their care and achieve a successful care outcome. Additionally, the care plan may be automatically generated and/or edited to include supportive services for addressing SDoH, helping to improve health disparities amongst individuals and promoting well-rounded patient care.

A machine learning model may additionally be used to predict and quantify a readmission risk for the patient based on their electronic health data. A care provider may be alerted to the readmission risk via the user interface, which may inform the care provider's decisions on their edits and additions to the patient care plan or may cause the care provider to reconsider discharging the patient altogether. In this manner, a care provider may take preventative action to reduce the patient's readmission risk, improving patient outcomes and preserving healthcare resources. A machine learning model may additionally identify unclaimed CPT codes from electronic health data, automating the claims process and capturing unclaimed revenues for healthcare providers. These functionalities may be supported by a sophisticated microservices-based system that allows for the secure transmission of sensitive health information while ensuring scalability, fault tolerance, and ease of deployment with external systems.

In the following description of the various embodiments, it is to be understood that the singular forms “a,” “an,” and the” used in the following description are intended to include the plural forms as well, unless the context clearly indicates otherwise. It is also to be understood that the term “and/of” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The structure for a variety of these systems will appear from the description below. In addition, the present invention is not described with reference to any particular programming language. It will be appreciated that a variety of programming languages may be used to implement the teachings of the present disclosure as described herein.

Systems and methods for generating personalized care plans will first be described, followed by an exemplary user interface for editing and displaying personalized care plans and for viewing healthcare information.

### Systems and Methods for Generating Personalized Care Plans

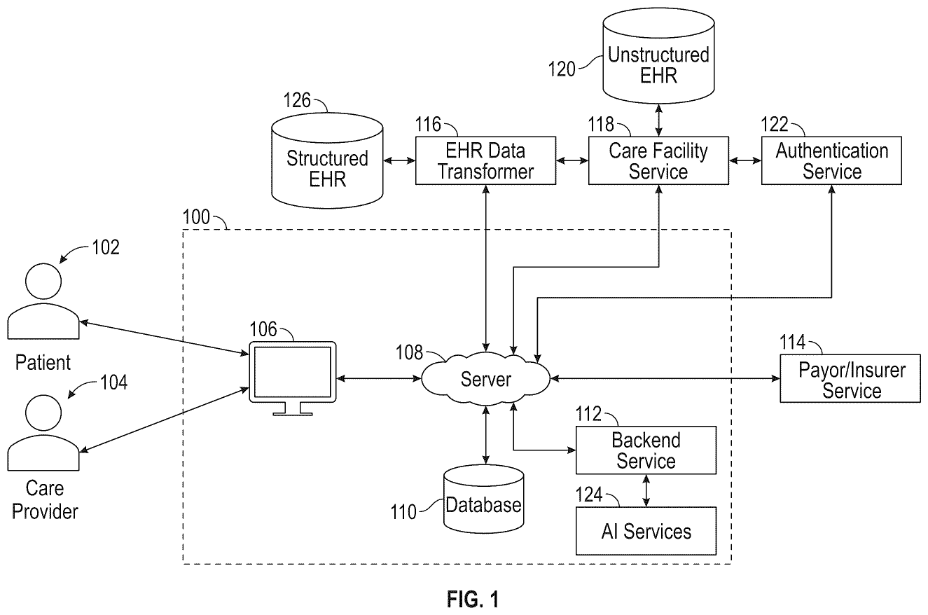

FIG. 1 illustrates an exemplary system 100 for generating personalized care plans, according to some embodiments. System 100 is designated with a dashed box in FIG. 1 and includes a client device 106, a server 108, a database 110, and backend service 112. As will be readily understood, one or more components in FIG. 1 that are shown as being external to system 100, may be included as part of the system 100 in some embodiments.

Additionally, system 100 may include, or may interact with, components having microservice architectures to allow for independent scalability, interoperability with external services, and fault tolerance. Each service may handle a specific task, such as care plan generation, patient data processing, or claims optimization. For example, system 100 in FIG. 1 is in communication with a payor and/or insurer service 114, an electronic health record (EHR) data transformer service 116, and an authentication service 122. Optionally, part or all of the backend service 112 and/or AI services 124 may have a microservices architecture.

Client device 106 may be used to display a frontend application accessible by a patient 102 and/or a care provider 104. Client device 106 may be, for example, a computer belonging to the user or service facility, a smartphone, or a tablet. Client device 106 may optionally have a touchscreen for touch interaction with the frontend application. The frontend application may include a user interface, as will be described in the next section. Client device 106 may allow a patient 102 and/or a care provider 104 to enter inputs into system 100 and to view and edit outputs from system 100, as will be described.

System 100 may also include a server 108 which may receive and transmit requests to/from the frontend application, the backend service 112, and the database 110 through API calls over a network. Server 108 may additionally receive and transmit requests through API calls to and from various services that may be external to the system, such as a care facility service 118. In some examples, server 108 is a cloud-based server hosted by a cloud service provider (e.g., Amazon Web Services, Google Cloud Platform, Microsoft Azure, etc.). In other examples, server 108 is an on-site or remote-hosted server. As will be described, requests for access to a care facility service 118 by server 108 may be reviewed and approved by an authentication service 122 to ensure proper data security. More generally, data storage and transmission to, from, and throughout system 100 may take place in any manner that complies with applicable electronic health data security regulations, such as HIPAA.

System 100 may additionally include one or more databases, e.g. database 110. In some examples, database 110 may be used to store information on various care facilities, including members of nationwide healthcare systems, smaller healthcare networks, and independent practitioners. Database 110 may be a NoSQL database, such as MongoDB, Amazon DynamoDB, or Cosmos DB for storing provider information in an unstructured or semi-structured format. Database 110 may also be a SQL database such as PostgreSQL, MySQL, MariaDB, or Oracle for storing structured information. In some examples, database 110 may be a vector database such as Pinecone, Milvus, Qdrant, Chroma, or MongoDB Atlas for storing provider information as vector embeddings that are searchable using semantic search techniques. As will be appreciated, any combination of SQL, NoSQL, and vector databases could be used in system 100, each storing different types of data. Care provider information may be stored in various structured or unstructured formats, such as plain text, markdown, HTML, XML, JSON, or any other formats supported by the type of database.

The data stored in database 110 may include, for example, the names, addresses, and contact information of the facilities, insurance providers accepted, the names and credentials of healthcare providers at the facility, whether they are accepting new patients, appointment availability, pricing, and Medicare and Medicaid information. Additionally, categories and descriptions of the various services and treatments offered at each facility may be stored in database 110. These categories and descriptions may include, for example, natural language keywords that may be used, optionally by a machine learning model such as a large language model (LLM), to query the database for care providers and facilities that match keywords found in the patient's health records. A non-exhaustive list of exemplary healthcare service categories and their corresponding keywords are provided in Table 1 below.

TABLE 1
Healthcare Service Categories and Keywords/Phrases

| Category | Keywords/Phrases |
|---|---|
| Wound care | Complicated wound, wound vac, wound care |
| Total parenteral | TPN, total parenteral nutrition, |
| nutrition | intravenous nutrition |
| IV infusion | IV antibiotic, chemotherapy, IV infusion |
| Memory Care | Alzheimer's disease, dementia, memory care |
| Dialysis | Peritoneal dialysis, hemodialysis, dialysis |
| Telemetry | Cardiac telemetry, radiotelemetry, telemetry |

In some examples, information on supportive service providers may be stored in a database such as database 110. This supportive services information may be stored in the same database as the healthcare provider information or in a separate database. Supportive services may be non-medical services that can help to promote healthcare equity and address the root causes for health disparities amongst patients of various socioeconomic backgrounds. These supportive services may address social determinants of health (SDoH). Supportive services can be included in a personalized care plan so that a patient receives well-rounded care, is set up for a successful care outcome, and has a lower chance of readmittance. In some examples, supportive services may be offered at healthcare service providers in addition to healthcare services. For example, a healthcare facility may offer transportation support for patients who lack access to reliable transit to get to their appointments. Supportive services may also be provided by supportive service providers that do not offer healthcare services. Food banks and community housing resources are examples of supportive services.

Information on supportive services offered at healthcare facilities, as well as supportive service providers that offer non-medical assistance, may be stored in database 110. The supportive services information may include, for example, the names, addresses, and contact information of the services providers, eligibility requirements and availability, and natural language descriptions of the services offered at each. As with the care provider information, the descriptions of the supportive services may include a category of the supportive service offered along with keywords that can be used to query the database for keywords found in the patient's health records. A non-exhaustive list of exemplary categories of supportive services and corresponding keywords and phrases are provided in Table 2 below.

TABLE 2
Supportive Services Categories and Keywords/Phrases

| Category | Keywords/Phrases |
|---|---|
| Health literacy | Problems related to health literacy, challenges |
| | in understanding health information |
| Housing | Homelessness, inadequate housing, temporary |
| | housing situations, housing instability |
| Food | Food insecurity, accepts food stamps, SNAP |
| | benefits, nutritional assistance |
| Transportation | Transportation insecurity, lack of access to |
| | public transportation, transportation |
| | affordability issues |
| Economic | Financially insecure, utility insecurity, |
| | financial resources, inability to afford |
| | essential utilities |

Besides keywords and phrases, medical services and supportive services may optionally be stored in database 110 along with their corresponding CPT codes, ICD10 codes, and/or Z-codes for querying by a large language model. Additionally, the lists in Tables 1 and 2 are non-exhaustive such that other categories and/or keywords and phrases may be included. For example, a “food as medicine” category may be included as a medical service category or supportive service category. Keywords and phrases in the “food as medicine” category may include produce prescription programs, medically tailored groceries, medically tailored meals, nutritional counseling and education, supplemental nutrition assistance, culturally tailored meal options, community-based food support, meal planning and shopping assistance tool, chronic disease prevention programs, and pediatric nutrition support. Additionally, a “care delivery” category may be included as a medical service category or supportive service category. Keywords and phrases in the care delivery category could include in-person office visits, telemedicine/telehealth, hospital-based care, concierge medicine, home visits, group or shared medical appointments, remote patient monitoring (RPM), chronic care management (CCM), bundled payments, value-based care, direct primary care (DPC), physician-led accountable care organizations (ACOs), collaborative and/or team-based care, facility and/or institution-based salaries, fee-for-services (FFS) models, or global payments accepted.

Referring still to FIG. 1, system 100 may include backend service 112. Backend service 112 may include server-side programming environments such as Node.js and Express, which may communicate with the frontend application to manage data requests, interactions with AI services 124, and integration with external systems. AI services 124 may include natural language processing (NLP) services such as PyTorch, TensorFlow, and HuggingFace that can be leveraged to train and fine-tune machine learning models to automatically generate personalized care plans from patient EHR, identify missing CPT codes, and translate patient care plans into multiple languages. In some examples, the AI services 124 may include one or more machine learning models. The machine learning models may be large language models (LLMs), such as Llama 3.x, GPT-4, GPT-4o, Gemini 1.5, or Claude 3.5. LLMs based on transformers (e.g. BERT, RoBERTa, GPT-4) may be used to translate ICD10 codes and ZCodes in patient EHR into corresponding natural language descriptions of medical conditions and SDoH for use in generating the patient care plan. One or more machine learning models of AI services 124 may be hosted on a private server and accessed behind a firewall to protect sensitive patient information. In some examples, AI services 124 includes multiple machine learning models, with each trained to perform a specific task. In other examples, a single machine learning model may be trained to perform multiple tasks. Accordingly, tasks that are described as being performed by separate machine learning models may optionally be performed by the same machine learning model, provided that the model is trained to perform both functions.

In some examples, a machine learning model in AI services 124 may be used to identify medical condition keywords in electronic health data associated with the patient. To perform this function, a machine learning model may be trained and/or fine-tuned from pairs of inputs and outputs that may include, for example, medical condition keywords paired with de-identified sets of real EHR. The de-identified sets of EHR may include international classification of diseases (ICD) codes (e.g. ICD-9, 10, or 11), which may identify diagnoses of medical conditions and procedures performed for each condition. Each code may be partitioned into sub-codes, which may include specific circumstantial details of the de-identified patient. The EHR training data can also include clinical reports records assigned to ICD-9 codes. The EHR training data can also include vital signs, medications, laboratory measurements, observations and notes charted by care providers, fluid balance, procedure codes, diagnostic codes, imaging reports, hospital length of stay, and survival data associated with the de-identified patients. In some examples, the de-identified EHR used to train the machine learning model may be sourced from publicly available medical datasets such as MIMIC-III. The EHR training data may include de-identified EHR obtained from a real patient or synthetic EHR generated for the purposes of training.

In addition to medical condition keywords obtained from de-identified EHR, a training output may also include a natural language description of treatments for each keyword. For example, a training output for “peritoneal dialysis” may state: “During peritoneal dialysis, a fluid called a dialysate is infused into the abdomen by a catheter, where it is left in the abdomen for a certain period of time and then drained. This process is then repeated.” By training the machine learning model from natural language descriptions of treatments, the machine learning model may be able to develop a substantive understanding of how certain medical conditions are treated, which can improve the quality of care plans generated by the machine learning model. For example, the machine learning model may generate a patient care plan having substantive information on each treatment option, which can help to inform the patient's decision-making.

Similar to medical condition keywords, a machine learning model may be trained to associate one or more SDoH keywords obtained from de-identified EHR with a corresponding Z-code for that particular SDoH. For example, a machine learning model may be trained with de-identified EHR training data including Z-code “Z59.812,” which corresponds to the SDoH keywords “housing instability.” The de-identified EHR may include information on how the care provider addressed this SDoH factor in the de-identified patient, for example, by recommending a community housing resource. Therefore, when the machine learning model is presented with a patient having “housing instability” or “Z59.812” in their electronic health data during use, the machine learning model will know to recommend a supportive services provider from database 110 that offers community housing services similar to those provided in the de-identified EHR from which it was trained.

A machine learning model may also be trained to identify CPT codes for treatments listed in a patient's electronic health records. For example, a machine learning model may be trained with inputs of services and treatments described in de-identified EHR paired with their corresponding CPT codes as outputs. A machine learning model may also be trained with de-identified EHR paired with exemplary claim submissions, including the anonymized patient information, CPT codes, ICD codes, and supporting documentation corresponding to the treatments described in the de-identified EHR. Further, a machine learning model may be trained to generate and/or translate patient care plans in multiple languages. The machine learning model may be trained from a multilingual text database which may include medical and non-medical keywords so that patient care plans can translated as needed to suit the language spoken by a care provider and/or patient.

Additionally, a machine learning model from AI services 124 may be trained to assess a readmission risk of a patient based on their EHR. For example, a machine learning model may be trained with inputs of de-identified EHR paired with one or more events in the EHR that contributed to the patient's readmission (e.g. lack of medication access, lack of compliance by the patient, post-treatment complications) to assess a patient's readmission risk from their electronic health data. A machine learning model may also be trained from medical condition and/or SDoH keywords paired with an associated readmission risk. This readmission risk may be provided as a relative degree of risk (e.g. low, medium, or high), or it may be provided as a numerical score that is calculated in accordance with an algorithm provided by a backend user. For example, a backend user may optionally train a machine learning model to score medical conditions and/or SDoH using an algorithm that weighs various factors derived from the electronic health data, such as the care provided during the most recent stay, the duration of most recent stay, current medications, and whether a condition is chronic or recurring.

Additionally, or alternatively, real care providers can be interviewed to gather training data to determine a readmission risk. For example, a care provider may be provided with sample EHR and may be asked to score and/or provide a relative degree of readmission risk for the patient, along with an explanation for their readmission risk assessment. The machine learning model can then be trained from this sample EHR paired with the determined readmission risk and the care provider's reasoning to learn which factors or events outlined in EHR tend to have the greatest influence on patient readmissions, so that the machine learning model may perform a similar analysis in an autonomous fashion. The readmission risk determined by the machine learning model can be used to inform the generation of the patient's care plan and may optionally be outputted to a user of the frontend application (e.g. care provider 104) so that they are prompted to consider alternative action instead of discharging or transferring the patient from their care. Optionally, the determination of a readmission risk may be performed without the use of artificial intelligence (e.g. through a non-AI numerical algorithm embodied in code for calculating readmission risks).

Training data obtained from real care provider interviews can also be used to train a machine learning model to generate an ordered list of recommended care providers based on electronic health data. For example, to gather training data to train a machine learning model, real care providers may be asked for their recommendation on how to treat a patient having one or more medical conditions indicated by keywords. The care provider's response may include a list of recommended care provider referrals, provided in an order from most to least recommended by the care provider, along with an explanation as to why each of the referrals were recommended in the particular order provided. The referrals may also be ranked or scored by the care provider to quantify their preference. A machine learning model may be trained with medical condition keywords paired with the ordered lists, rankings and explanations provided by the care provider for treating each condition. In this manner, a machine learning model can learn to recommend care providers and output an ordered list of care providers from most to least recommended in a similar manner as a real care provider.

Thus, a machine learning model may be trained to recommend a healthcare provider and/or a supportive service provider to a patient by matching medical condition, medical treatment, and/or SDoH keywords found in patient EHR with keywords found in healthcare provider and/or supportive service provider information stored in database 110. The machine learning model may output these recommended care providers in an ordered list from most to least recommended. Multiple recommendations for healthcare providers and/or supportive services providers may be integrated together to generate a personalized care plan for the patient.

In some examples, a machine learning model may be fine-tuned to filter or sort through a list of multiple recommended healthcare and/or supportive services providers based on one or more practical constraints configured by a backend user. For example, a machine learning model may be fine-tuned to filter a list of healthcare and/or supportive services providers down to those within a certain geographic radius of the patient's zip code, or only those healthcare providers who accept the patient's insurance. Other practical constraints could include cost, appointment availability, and level of services offered (e.g. short-term care, long-term care, intensive care, etc.). These practical constraints on care plan generation may be provided to the machine learning model through a prompt or input entered by a frontend user (e.g. by the selection of a filter to reflect a certain geographic radius or a certain insurance provider in the user interface), or as a system prompt configured by a backend user, as will be described.

A machine learning model may be trained and/or fine-tuned to generate personalized care plans for a patient using supervised learning to ensure that the generated plans are realistic and accurately reflect the patient's needs. For example, a machine learning model may be trained using a classification algorithm for categorizing electronic health data based on keywords and phrases. Additionally, a machine learning model may be trained using a regression algorithm to predict readmission risk. A backend user may supervise the training process manually by comparing a machine learning model-generated care plan output to EHR training data to determine whether the generated care plan adequately addresses the patient's needs indicated by the EHR. Additionally, a machine learning model's care plan output can be validated against a care plan created by a real care provider to verify that the model's outputs are realistic and accurate. If the care plan is inaccurate or omits services that the patient needs, the backend user may provide this feedback directly to the machine learning model or may adjust the machine learning model's system prompt to correct the inaccuracies or to account for the omitted services. The training process for generating automated claims based on CPT codes, for scoring readmission risk, and for multilingual care plan generation can be supervised in a similar manner. Further, a frontend user, such as a care provider or a patient, may optionally input feedback on the care plan into a user interface, and this feedback can be used to fine-tune a machine learning model based on real user experiences.

With respect to FIG. 1 generally, the flow of information through system 100 may be as follows. A healthcare provider 104 may initiate the generation of a care plan for a patient 102 using the frontend application displayed at client device 106. For example, healthcare provider 104 may input their access credentials and may indicate their selection of patient 102 for care plan generation, for example, by selecting a visual affordance in the user interface associated with patient 102 (e.g. a button on the patient's profile). Server 108 may then transmit a request for access to authentication service 122, which may include the access credentials entered by care provider 104 into the user interface. Authentication service 122 may be Amazon Cognito, Okta, FusionAuth, Keycloak, or any other authentication service suitable for restricting access to EHR. In some examples, authentication service 122 may restrict access to EHR using role-based access control (e.g. by limiting access to patient 102's data depending on the care provider 104's role in a hospital system).

If the care provider 104 is authorized to access the EHR, authentication service 122 may grant the server 108's request, allowing the server to access unstructured EHR data 120 via a care facility service 118. In some examples, a frontend user may transmit requests for certain information from a patient's EHR rather than receiving the patient's unstructured health records in their entirety. For example, care facility service 118 may be a fast healthcare interoperability resources application programming interface (FHIR API), such as Epic on FHIR, Google Cloud Healthcare API, Azure API, or Akana. HL7 FHIR is a standard for exchanging healthcare information across different platforms in a secure manner. FHIR is a resource-based framework, with each resource defining component data elements, constraints on data, and data relationships that allow patient records to be exchanged. A frontend user may request certain categories of FHIR resources from the care facility service 118 (e.g. clinical notes, patient-entered questionnaires, radiology results, and so on). In some examples, care facility service 118 may retrieve this information in an unstructured format from unstructured EHR data 120.

To ensure that the unstructured data is converted into a format that the machine learning models in AI services 124 can understand, the unstructured data may be processed by an EHR data transformer 116 which may be an HL7 FHIR transformer. EHR data transformer 116 may convert the EHR data into HL7 FHIR R4 format and store it as structured EHR 126 to be used by the backend service 112. The structured EHR 126 may similarly be stored and accessed through a FHIR API, such as Amazon Healthlake, Epic on FHIR, or Google Cloud Healthcare API. In some examples, a FHIR API may include a built-in FHIR transformer, such that EHR data transformer 116 is a FHIR API (e.g. Amazon Healthlake, Epic on FHIR, or Google Cloud Healthcare API). The structured EHR 126 may be stored in any format supported by FHIR (e.g. XML, JSON, and RDF). Optionally, electronic health information received through the frontend application 106 (e.g. updates to patient demographics, medications, etc.) may be transmitted by server 108 to EHR data transformer to be formatted in a FHIR format and stored as structured EHR 126.

The structured EHR 126 may include electronic health data such as demographics information such as the patient's name, birthdate, gender, and address. The electronic health data may also include clinical observations, such as the patient's height, weight, blood pressure, heart rate, respiratory rate, and appointment notes or charts entered by a care provider. The electronic health data may also include active and inactive medical conditions of the patient, current medications and dosage instructions, and information about the patient's current care providers and insurance providers.

Server 108 may receive patient 102's electronic health data in HL7 FHIR R4 format from EHR data transformer 116 and may transmit the structured electronic health data to backend service 112. The backend service 112 may select a first machine learning model from AI services 124 to receive the electronic health data as an input. As mentioned above, in some examples, a frontend user and/or a system prompt may specify certain FHIR resource categories (e.g. clinical notes, patient-entered questionnaires, and radiology results) to be inputted into the machine learning model so that the machine learning model is not overwhelmed with irrelevant context. Based on the electronic health data, the machine learning model may identify keywords (e.g. medical condition, medical treatment, and/or SDoH keywords) to be used in searching database 110. A second machine learning model may receive the identified keywords and may transmit a request over server 108 for provider information stored in database 110 that matches the keywords from the electronic health data in the FHIR format. In addition to keywords, the machine learning model may analyze the electronic health data for the patient's location information (e.g. address and/or zip code) and their insurance provider(s). The machine learning model may transmit a request over server 108 to database 110 for care providers located within a certain geographic radius of the patient's zip code and/or for care providers who accept the patient's insurance. The machine learning model may consider the care provider's location and whether they accept the patient's insurance in determining whether to recommend the care provider. The second machine learning model may optionally apply learned constraints (e.g. cost constraints, location constraints, insurance provider constraints, appointment availability constraints) to narrow down the care providers retrieved from database 110. Then, the second machine learning model may rank or order the recommended care providers in the manner learned from observing real care provider preferences during training. The ordered list of recommended care providers outputted by the second machine learning model may optionally be displayed at a user interface, as will be described.

In some examples, the machine learning model may receive a prompt from a frontend user with natural language instructions on how to generate and rank the list of recommended care providers. These instructions may be entered by a frontend user into the user interface. In other examples, a system prompt from backend service 112 may be provided to a machine learning model from AI services 124 with instructions and constraints to follow in generating the list of recommended care providers. For example, a backend user may engineer a system prompt for a machine learning model which may state, for example: “Your job is to provide a list of all healthcare and supportive services providers offering services that match with the patient's needs, along with descriptions of the services offered, their locations, and their contact information. Include only those service providers that are within a 50-mile radius of the patient, that accept the patient's insurance, and that have appointment availability within the next month.” This prompt may be provided by the backend service 112, along with the structured electronic health data 126 fetched by server 108 as part of the input into the machine learning model.

As will be described, the list of recommended care providers may serve as a first output from the second machine learning model, and this output may be refined by a user's selections in a user interface using a “human-in-the-loop” approach. The second machine learning model may then generate a personalized, integrated care plan for patient 102 once the user's selections have been made. The integrated care plan may include a list of recommended providers and their corresponding information (e.g. location, hours, services offered, and the like) as well as a natural language explanation as to why each of the care providers were recommended. This natural language explanation may be sourced from the care provider information stored in database 110, and/or the explanation may be generated by the second machine learning model as learned from the care provider interviews during training. The integrated care plan may be provided in a text-based format, such as JSON or XML, to backend service 112. Alternatively, the care plan output may be routed to a third machine learning model for translating the patient care plan into one or more languages (e.g. from English to Spanish and/or French) before it is provided to backend service 112. The backend service 112 may then transmit the generated (and optionally translated) patient care plan to the frontend application, where it may be converted into a visual format to be displayed at client device 106 for viewing by patient 102 and/or care provider 104.

Additionally, or alternatively, backend service 112 may select a machine learning model from AI services 124 that can receive patient 102's electronic health data as an input. As with the care plan generation, the input may also include a prompt which may provide instructions to the machine learning model on submitting a claim to a payor/insurer 114 as an output. A prompt configured by a backend user may state, for example: “Your job is to generate a claim to the patient's insurance provider for the services rendered in the patient's most recent appointment. Identify all possible CPT codes for the services described in the electronic records of the patient's most recent appointment. Provide the draft insurance claim and the electronic records of the patient's most recent appointment as an output.”

Based on the prompt, the machine learning model may identify CPT codes from records of healthcare services and treatments performed on the patient that are reflected in the retrieved electronic health data. Using the identified CPT codes and patient information reflected in the electronic health data, the machine learning model may generate a claim for a payor/insurer 114. The claim may be provided to backend service 112, which may communicate via an API call to the payor/insurer service 114 to submit the claim and coordinate claim processing and claim appeals. By using a machine learning model to identify CPT codes, this can reduce the number of revenue opportunities that go unclaimed while streamlining the claim and appeal process for care providers and patients. As mentioned above, although the keyword identification, care provider recommendation and care plan generation, multilingual translation, and CPT code identification functions are described as being performed by separate machine learning models, in some examples, one or more of these functions, or optionally all of these functions, may be performed by the same machine learning model.

FIG. 12 illustrates a device 1200, which may be part of system 100 or may host one or more services internal or external to system 100. Client device 106 may include some or all of the components of device 1200. Additionally, device 1200 may be used to implement some or all steps of the methods described herein. device 1200 can be a host computer connected to a network. Device 1200 can be a client computer or a server. As shown in FIG. 12, device 1200 can be any suitable type of microprocessor-based device, such as a personal computer, workstation, server, or handheld computing device (i.e., a portable electronic device) such as a phone or tablet. The device can include, for example, one or more of processors 1202, input device 1206, output device 1208, storage 1210, and communication device 1204. Input device 1206 and output device 1208 can generally correspond to those described above and can either be connectable or integrated with the computer.

Input device 1206 can be any suitable device that provides input, such as a touch screen, keyboard or keypad, mouse, or voice-recognition device. Output device 1208 can be any suitable device that provides output, such as a touch screen, haptics device, or speaker.

Storage 1210 can be any suitable device that provides storage, such as an electrical, magnetic, or optical memory, including a RAM, cache, hard drive, or removable storage disk. Communication device 1204 can include any suitable device capable of transmitting and receiving signals over a network, such as a network interface chip or device. The components of the computer can be connected in any suitable manner, such as via a physical bus or wirelessly.

Software 1212, which can be stored in storage 1210 and executed by processor 1202, can include, for example, the programming that embodies the functionality of the present disclosure (e.g., as embodied in the systems and methods as described above).

Software 1212 can also be stored and/or transported within any non-transitory computer-readable storage medium for use by, or in connection with, an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1210, that can contain or store programming for use by, or in connection with, an instruction execution system, apparatus, or device.

Software 1212 can also be propagated within any transport medium for use by, or in connection with, an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by, or in connection with, an instruction execution system, apparatus, or device. The transport-readable medium can include, but is not limited to, an electronic, magnetic, optical, electromagnetic, or infrared wired or wireless propagation medium.

Device 1200 may be connected to a network, which can be any suitable type of interconnected communication system. The network can implement any suitable communication protocols and can be secured by any suitable security protocols. The network can comprise network links of any suitable arrangement that can implement the transmission and reception of network signals, such as wireless network connections, T1 or T3 lines, cable networks, DSL, or telephone lines.

Device 1200 can implement any operating system suitable for operating on the network. Software 1212 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

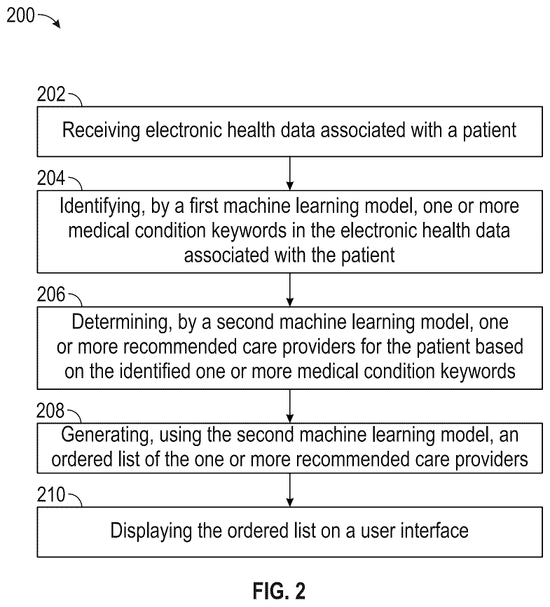

FIG. 2 illustrates an exemplary computer-implemented method 200 for generating patient care plans, according to some embodiments. Step 202 may include receiving electronic health data associated with a patient. This step may entail, for example, transmitting a request for access to an authentication service 122 (e.g. Amazon Cognito) and receiving access to unstructured EHR 120 stored by a care facility service 118 as shown in FIG. 1. Step 204 may include identifying, using a first machine learning model, one or more medical condition keywords in the electronic health data associated with the patient.

Step 206 may include determining, by a second machine learning model, one or more recommended care providers for the patient based on the identified medical condition keywords. This may involve searching a database storing care provider information using the identified one or more medical condition keywords. For example, the second machine learning model may transmit a request to server 108 for care provider information stored in database 110 that contains the identified keywords, and server 108 may transmit data corresponding to the retrieved providers to the second machine learning model. The second machine learning model can apply any learned and/or prompted constraints to filter the search results (e.g. narrowing down to only the resulting providers that accept the patient's insurance, are within a certain geographic radius of the patient, and/or have immediate appointment availability) and to order and/or rank the care providers in an order from most to least recommended. At step 208, the second machine learning model may then generate an ordered list of the recommended care providers determined at step 206. At step 210, the ordered list of recommended care providers may be displayed at a user interface (e.g. the frontend application). This user interface will now be described in the following section.

### Exemplary User Interface for Generating Personalized Care Plans

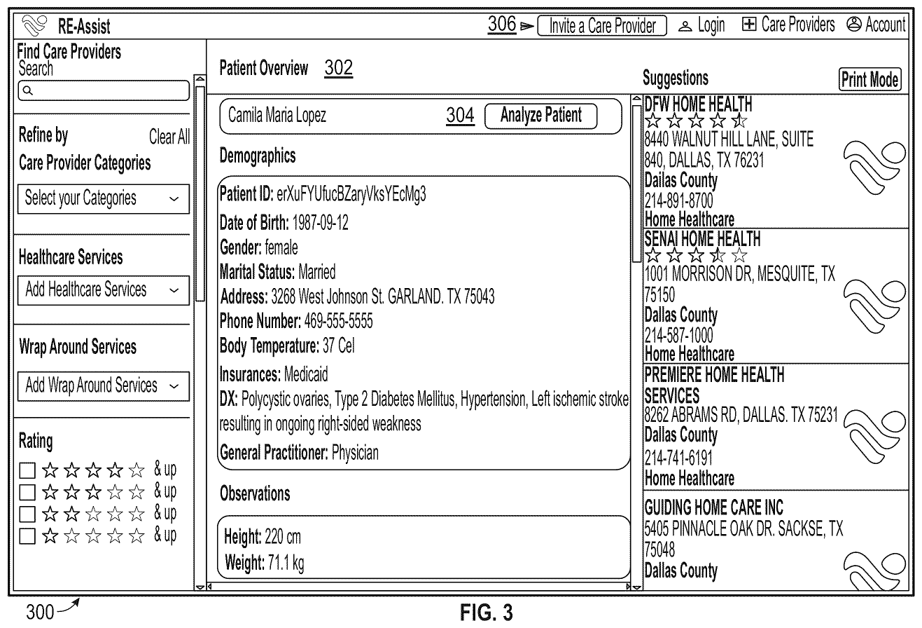

As mentioned in the previous section, in some embodiments, a user interface for the frontend application is provided that allows a patient and/or care provider to view and interact with patient data, generate and edit patient care plans, and monitor care outcomes. FIG. 3 illustrates a patient overview page 302 in user interface 300. When the frontend user is a patient, the patient may optionally be able to view and edit the information that is displayed on their patient overview page 302. The information on the patient overview page 302 may include a unique patient identifier, their date of birth, marital status, location information (e.g. address and zip code), contact information (e.g. phone number and/or email address), insurance information, physical traits (e.g. height, weight, most recent vitals), and their current medical conditions.

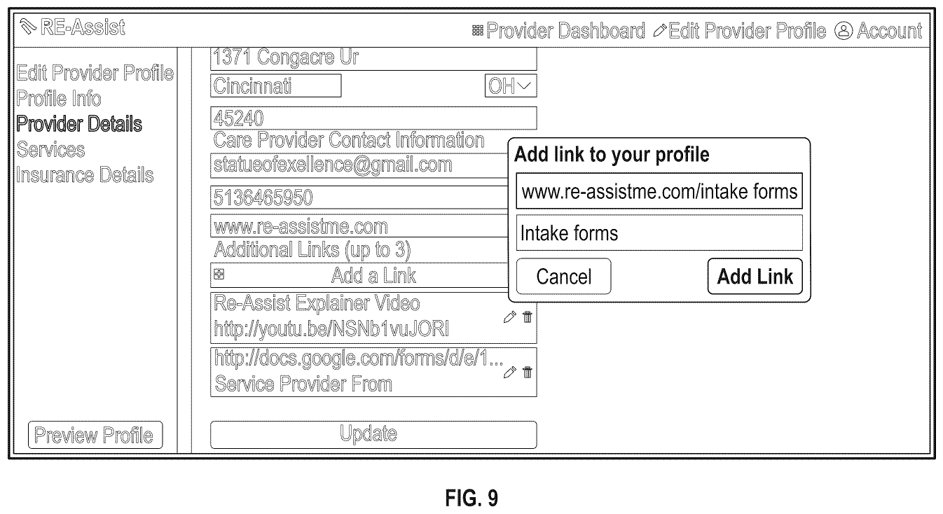

A patient may be able to view their overview page 302 and make edits and updates, optionally upon request and approval from the care provider. In addition to the patient overview page 302, the user interface 300 may display a search bar for the patient to locate care providers, dropdown menus for filtering the resulting care providers by category, services offered, and/or rating. A list of suggested care providers may optionally be displayed in a side bar of the user interface 300 for convenient access. As shown in FIG. 9, care providers may also view and edit their own profiles to include contact information, intake forms, insurance details, and health literacy resources.

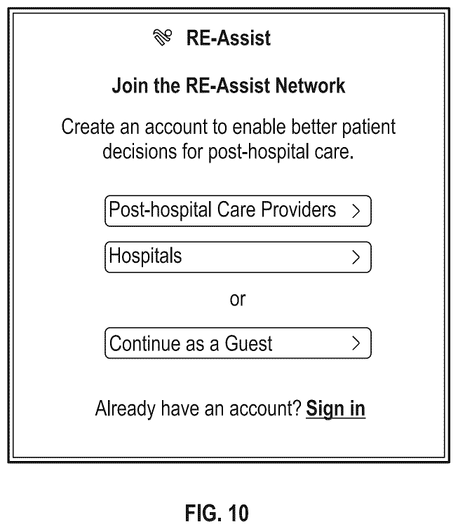

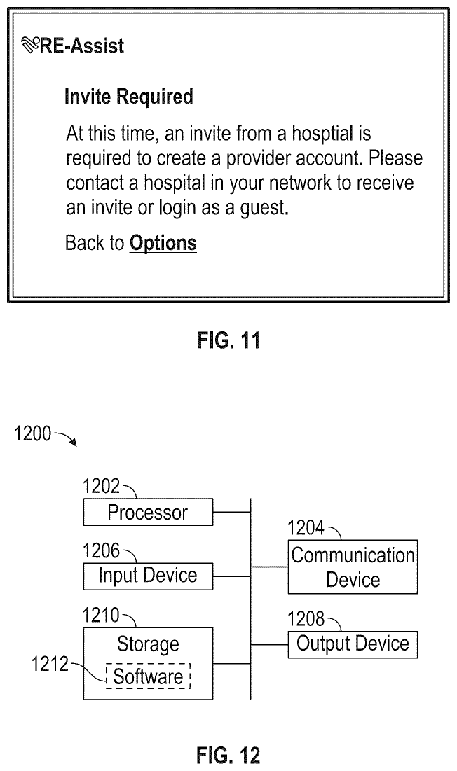

Referring back to FIG. 3, user interface 300 may include an icon 306 for inviting a care provider to create an account on the frontend application. The care provider may be sent an invite via email, text message, or other suitable communication. In some examples, as shown in FIG. 11, the frontend application may be configured such that receiving an invite is the only way to create a provider account, which can help to prevent data privacy breaches and fraud. The invite may optionally include a link for creating an account, and selection of the link may open an account creation page as shown in FIG. 10. As shown, post-hospital care providers and hospitals may create an account on this page. In some examples, a “guest” mode may be supported for limited access to view care plans and provider resources.

Certain features of user interface 300 may be visible in both a patient view and a care provider view. For example, both a care provider and a patient may be able to view and select icon 306 for inviting a care provider to create an account. Other features may only be accessible by a care provider. For example, in FIG. 3, patient overview page 302 may depict a “provider view,” such that an icon for analyzing the patient 304 to generate a personalized care plan may only be visible and accessible to a care provider with proper access credentials. In some examples, receipt of a selection of the icon for analyzing the patient 304 by the backend services may cause the backend services to request the electronic health data associated with the patient and provide the requested electronic health data to a machine learning model of AI services 124 so that a list of suggested providers is automatically generated based on the electronic health data.

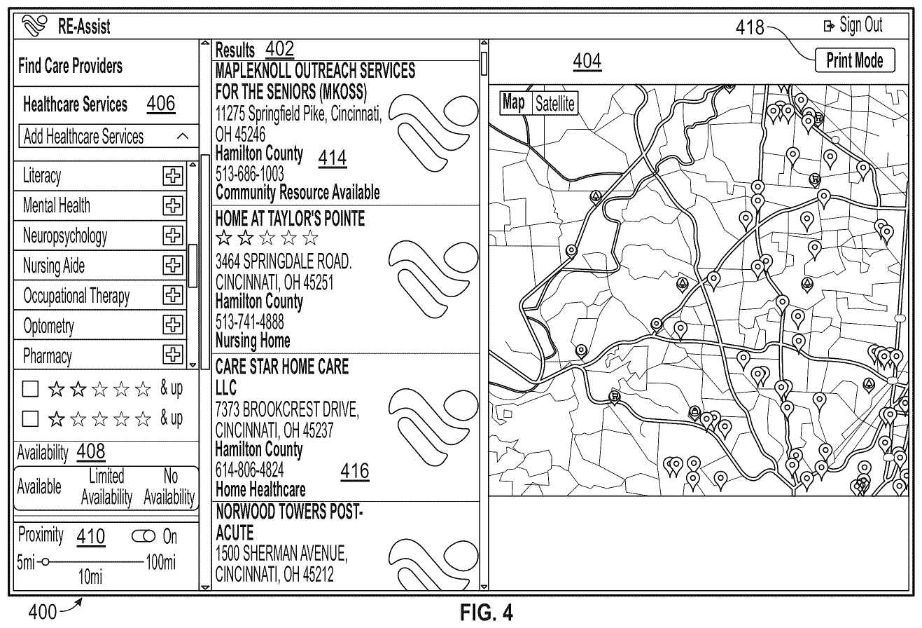

FIG. 4 illustrates an exemplary view of a provider list 402 in a user interface 400. In some examples, provider list 402 may be displayed automatically after the selection of the icon for analyzing the patient 304. Provider list 402 may initially display recommended providers resulting from a machine learning model's analysis of the patient's electronic health records, as described with respect to system 100 and method 200. Provider list 402 may include healthcare providers as well as supportive services providers that are recommended for the patient in an order from most to least recommended as determined by the machine learning model. For example, provider 414 offers outreach services and community resources for seniors, which can be considered supportive services. Provider 416 offers in-home healthcare, which can be consider a healthcare service. The provider list 402 may display each recommended provider, along with their location information (e.g. address and county), their contact information, the category of services provided, their rating, and their appointment availability. Additionally, an interactive map 404 may be provided that depicts the locations of the recommended providers, allowing a user to select and drag the map to change the viewing area, to zoom into a particular geographic area, and to view information for a particular care provider by selecting their corresponding map pin.

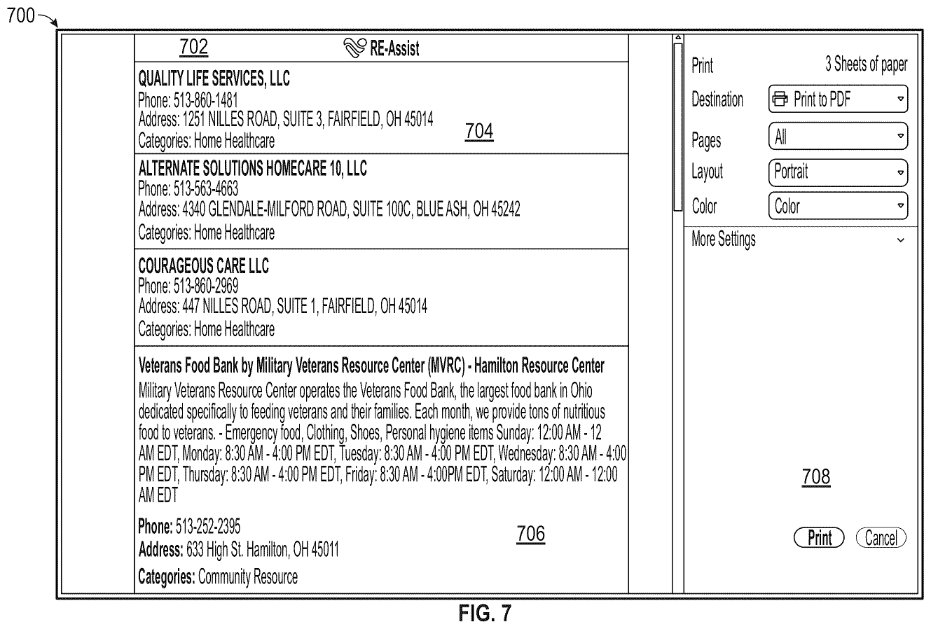

A user may be able to edit the recommended list of care providers using the user interface 400. For example, one or more dropdown menus 406 may be provided to allow a user to select and deselect various primary care services for filtering the recommended care providers in the provider list 402. A user may also be able to filter the recommended care providers by selecting an appointment availability filter 408. Further, a user may be able to filter the recommended care providers by proximity to the patient, and this option is shown by way of example as a slider icon 410. As the user makes these selections, the frontend application may communicate the user selections to the backend services, so that the machine learning model may update the displayed order of recommended care providers in real time. As will be described with respect to FIG. 7, a user may be able to consolidate the list of recommended care providers into an integrated care plan by selecting a print mode icon 418.

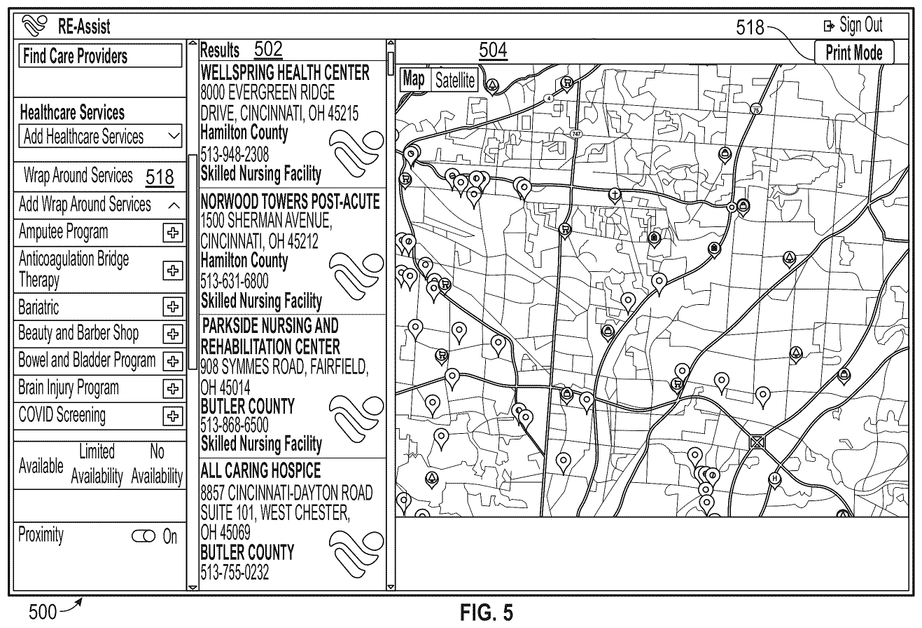

FIG. 5 shows an example of a provider list 502 displayed in a user interface 500. The provider list 502 and the interactive map 504 may be displayed in the same or substantially the same manner as described with respect to FIG. 4. Additionally, FIG. 5 shows a dropdown menu 518 for selecting “wrap around services” in addition to the primary services shown in the dropdown menu above it. As used herein, the term “wrap around services” includes non-medical services, medical services, and supportive services that may be lower in priority or necessity than the treatment services for a patient's primary condition(s). In some examples, wrap around services may include services that are based on patient preferences rather than medical or social need. As shown, the dropdown menu 518 for selecting wrap around services includes providers offering specialized medical services such as amputee programs and anticoagulation bridge therapy, as well as non-medical service providers such as beauty and barber shops to enable a patient to continue their self-care practices while obtaining medical treatments to support their mental health. By enabling the selection of wrap around services for inclusion into a patient care plan, the patient care plan is made highly customizable, promoting improved patient compliance and supporting beneficial treatment outcomes.

The filters displayed in the dropdown menus 406, 518 for filtering care provider results based on primary and/or secondary services may be customized to fit the needs of the particular patient using a machine learning model. For example, the machine learning model used to identify medical condition keywords in patient data may also be used to identify SDoH keywords and/or patient preferences indicated in their electronic health data. The machine learning model may compile the medical condition keywords for primary and secondary conditions of the patient (including both medical and non-medical conditions) and may populate the dropdown menus in the user interface (e.g. 406, 518) with filters that personalized to the needs and preferences of the patient. Accordingly, different needs of different patients may result in different primary and/or secondary service filters being displayed in the dropdown menus for filtering the care provider recommendations. This provides a customized user experience for the patient and the patient's care provider and provides a more precise and meaningful way to filter through the care provider results recommended by the machine learning model.

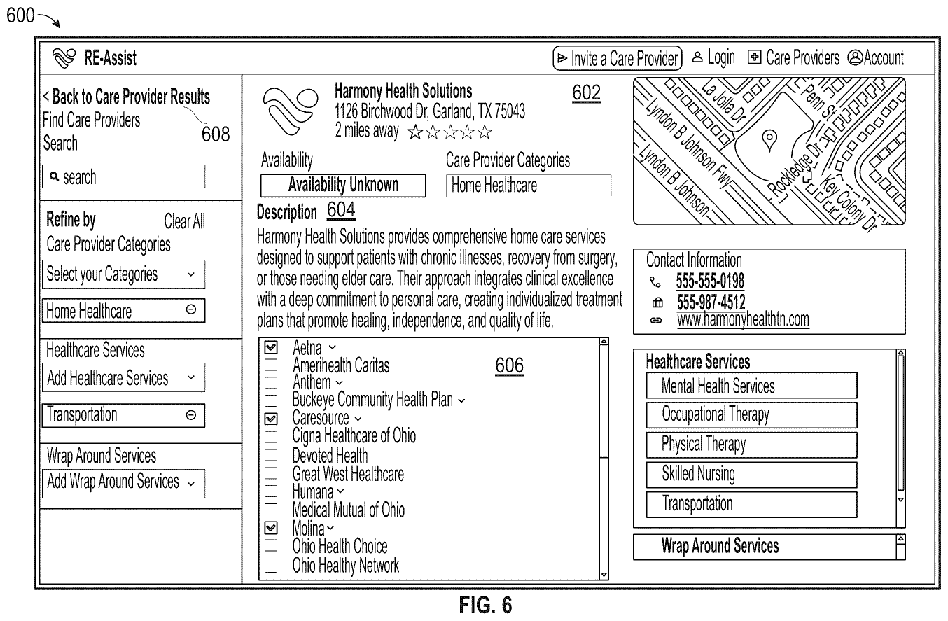

FIG. 6 illustrates an exemplary care provider page 602 of a user interface 600. The care provider page 602 may be shown upon the user selection of a recommended care provider in a provider list (e.g. 402, 502). The provider page 602 includes a description 604 of the care provider's service offerings. This description 604 may optionally be generated by a machine learning model based on care provider information obtained from database 110 of system 100. This description 604 may optionally be provided through a user input at the user interface, for example, using the provider profile editing page shown in FIG. 9. Additionally, a list 606 of common insurance programs may be provided with checked boxes indicating that the care provider accepts the particular insurance program. A user may then return to the provider list 402, 502 by selecting a “back” button 608.

As mentioned in the previous section, a machine learning model of AI services 124 may be trained to predict a readmission risk of the patient. Optionally, a machine learning model may be provided with the user's edits to the provider list in real time. The machine learning model may update the predicted readmission risk of the patient as the user makes the edits to the provider list. If the user makes an edit that will adversely affect the readmission risk, an alert may be displayed in the user interface indicating as such, so that the user is prompted to reconsider making the change. Optionally, if the readmission risk of a patient is high at the onset of care plan generation, an alert may be displayed in the user interface upon the initial display of the provider list so that a care provider is prompted to reconsider discharging the patient. This can help to prevent readmission and improve the chance of a successful patient outcome. An indication of the patient's readmission risk may optionally be displayed as part of their comprehensive care plan, as will be described.

Returning to FIGS. 4 and 5, once the user has made their desired revisions to the provider list 402, 502, the user may select an icon 418, 518 for changing the display from the list view to generate the integrated care plan. Optionally, this may be a “print mode” icon such that selection of the icon generates a print preview of the integrated care plan. An exemplary print preview 700 is shown in FIG. 7. As shown, the integrated care plan 702 may include the list of recommended providers, filtered and sorted to account for the user's selections. The integrated care plan 702 may include the names and categories of the care providers, including both healthcare providers 704 and supportive services providers 706, their contact information, and their location information. As described above, the integrated care plan 702 may also include a natural language explanation as to why each care provider was recommended, which may be generated by a machine learning model.

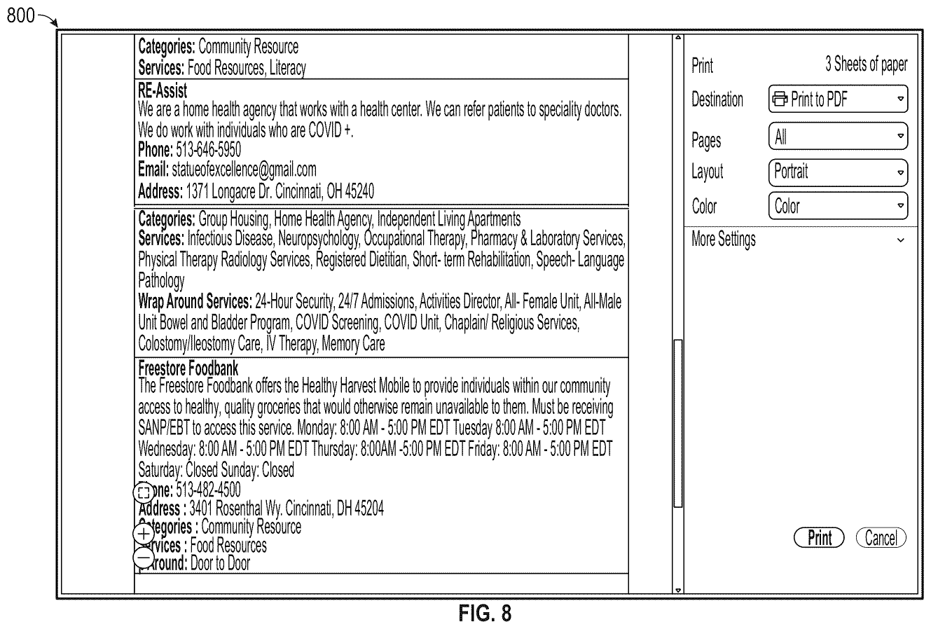

As shown in FIG. 8, an integrated care plan 802 may also describe the primary services offered (e.g. infectious disease, neuropsychology, occupational therapy) as well as the wrap around services offered (e.g. 24-hour security and admissions, activities, care units composed of a certain gender identity). Therefore, a user can view all of the information that they may need to make informed decisions about their care in a consolidated format that is uniformly organized and easily understood, regardless of the user's health literacy. This can provide a drastic improvement over existing discharge practices, where the care plan is typically provided as a disorganized bundle of paperwork that a patient may not understand or that may not comprehensively address all of the patient's needs. The user may optionally print the integrated care plan 702 by selecting a print icon 708.

As previously described, the user interface may be used by a patient, a care provider, or both. As will be readily understood, it may be desirable to have only certain features available to care providers with proper access credentials, as the review of and the revisions to the generated care plan may require medical expertise and experience. Accordingly, the options described for editing and filtering the lists of recommended providers, such as the dropdown menu 406 for selecting primary care services, may only be displayed in a “provider view” of the user interface in some embodiments. Alternatively, a patient may be able to propose edits to their care plans themselves using the same features as present in the provider view, but these changes may require the approval of the care provider before they are reflected in the comprehensive care plan. In other examples, the user interface may be provided to the patient in a “read only mode,” such that certain displayed features are visible but may not be selected by the patient.

Further, although not shown in the figures, a patient and/or care provider may input feedback on the generated care plan into the user interface. This inputted feedback may describe the ease of patient compliance, the overall care outcome, and suggestions on how the care plan could be changed to improve the patient outcome. The inputted feedback may also include details of a patient's positive, neutral, or negative experience with a particular provider. One or more of the machine learning models in AI services 124 may be fine-tuned from this feedback, improving the quality of generated plans and therefore improving patient care outcomes. Feedback on other features, such as on the accuracy of a machine learning model's prediction of a patient's readmission risk and on a machine learning model's identification of missing CPT codes, may be provided using the user interface. This feedback may be provided by the user in the form of natural language (e.g. spoken or typed feedback), numerical feedback (e.g. a numerical score), or it may be provided by the user's selection of visual elements displayed in the user interface (e.g. a selection of a number of star icons for rating the care plan, or a selection of a “thumbs up” or “thumbs down” icon).

A user interface may include additional capabilities and functionalities other than those shown and described with respect to FIGS. 3-11. For example, a user interface may be used to generate and display detailed reports on patient outcomes, care plan effectiveness, and hospital performance metrics based on the inputted user feedback described above. Additionally, reports may be generated and displayed that includes unclaimed CPT codes, optionally determined by a machine learning model, as well as revenue generated from subsequent appeals. The user interface may also be used to collect data on patient satisfaction, readmission rates, and care plan adherence, and reports that include these metrics may be generated and displayed at the user interface. Furthermore, the user interface may be used to automate workflows by managing appointment scheduling, supporting telehealth visits, and facilitating prescription refills and medication reminders. In this manner, the systems, methods, and user interfaces provided can help to streamline the care process for both patients and care providers, improving access to care, ease of compliance, and overall patient outcomes.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A computer-implemented method comprising:
receiving electronic health data associated with a patient;
identifying, by a first machine learning model, one or more medical condition keywords from the electronic health data associated with the patient,
wherein the first machine learning model is trained to identify medical condition keywords from training data comprising de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients;
determining, by a second machine learning model, one or more recommended care providers for the patient based on the identified one or more medical condition keywords;
generating, using the second machine learning model, an ordered list of the one or more recommended care providers,
wherein the second machine learning model is trained to generate the ordered list from training data comprising medical condition training keywords paired with recommendations by care providers of other care providers treating one or more of the medical conditions indicated by the training keywords; and
displaying the ordered list of the one or more recommended care providers on a user interface.

2. The computer-implemented method of claim 1, further comprising displaying an interactive map on the user interface indicating locations of the one or more recommended care providers.

3. The computer-implemented method of claim 1, further comprising:
determining a first set of filters for filtering the ordered list by one or more primary services, and a second set of filters for filtering the ordered list by one or more secondary services; and
displaying a first dropdown menu with the first set of filters and a second dropdown menu with the second set of filters on the user interface.

4. The computer-implemented method of claim 3, further comprising:
receiving, at the user interface, a user selection comprising one or more primary services from the first dropdown menu, one or more secondary services from the second dropdown menu, or both; and
updating the ordered list of the one or more recommended care providers based on the user selection.

5. The computer-implemented method of claim 3, wherein the one or more primary services, the one or more secondary services, or both comprise literacy support services, housing support services, transportation support services, food security resources, financial support resources, or any combination thereof.

6. The computer-implemented method of claim 1, wherein the method further comprises:
determining, by a third machine learning model, a readmission risk for the patient based on the electronic health data,
wherein the third machine learning model is trained to determine a readmission risk from training data comprising de-identified electronic health records paired with one or more readmission-causing events; and
displaying an indication of the readmission risk for the patient on the user interface.

7. The computer-implemented method of claim 1, further comprising:
identifying, by a fourth machine learning model, one or more current procedural technology (CPT) codes in the electronic health data associated with the patient,
wherein the fourth machine learning model is trained to identify the one or more CPT codes from training data comprising de-identified health records paired with corresponding CPT codes, and
generating, by the fourth machine learning model, an insurance claim based on the one or more CPT codes.

8. The computer-implemented method of claim 1, further comprising:
receiving, at the user interface, a user selection of a visual affordance for generating an integrated care plan;
in response to receiving the user selection of the visual affordance, generating, using the second machine learning model, an integrated care plan for the patient,
wherein the integrated care plan comprises the ordered list of the one or more recommended care providers and a natural language explanation for why each of the one or more recommended care providers were recommended; and
displaying the integrated care plan on the user interface.

9. The computer-implemented method of claim 8, wherein the integrated care plan comprises, for each of the one or more recommended care providers, a care provider location, services offered, insurance providers accepted, cost information, or any combination thereof.

10. The computer-implemented method of claim 1, further comprising, prior to receiving electronic health data associated with a patient:
receiving unstructured electronic health information associated with the patient; and
converting, using a fast healthcare interoperability resources application programming interface (FHIR API), the unstructured electronic health information into electronic health data associated with the patient, the electronic health data comprising a FHIR data format.

11. A system comprising:
one or more processors, and
memory storing computer program code executable by the one or more processors to cause the system to:
receive electronic health data associated with a patient;
identify, by a first machine learning model, one or more medical condition keywords from the electronic health data associated with the patient,
wherein the first machine learning model is trained to identify medical condition keywords from training data comprising de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients;
determine, by a second machine learning model, one or more recommended care providers for the patient based on the identified one or more medical condition keywords;
generate, using the second machine learning model, an ordered list of the one or more recommended care providers,
wherein the second machine learning model is trained to generate the ordered list from training data comprising medical condition training keywords paired with recommendations by care providers of other care providers treating one or more of the medical conditions indicated by the training keywords; and
display the ordered list of the one or more recommended care providers on a user interface.

12. The system of claim 11, wherein the system is further caused to display an interactive map on the user interface indicating locations of the one or more recommended care providers.

13. The system of claim 11, wherein the system is further caused to:
determine a first set of filters for filtering the ordered list by one or more primary services, and a second set of filters for filtering the ordered list by one or more secondary services; and
display a first dropdown menu with the first set of filters and a second dropdown menu with the second set of filters on the user interface.

14. The system of claim 13, wherein the system is further caused to:
receive, at the user interface, a user selection comprising one or more primary services from the first dropdown menu, one or more secondary services from the second dropdown menu, or both; and
update the ordered list of the one or more recommended care providers based on the user selection.

15. The system of claim 13, wherein the one or more primary services, the one or more secondary services, or both comprise literacy support services, housing support services, transportation support services, food security resources, financial support resources, or any combination thereof.

16. The system of claim 11, wherein the system is further caused to:
determine, by a third machine learning model, a readmission risk for the patient based on the electronic health data,
wherein the third machine learning model is trained to determine a readmission risk from training data comprising de-identified electronic health records paired with one or more readmission-causing events; and
display an indication of the readmission risk for the patient on the user interface.

17. The system of claim 11, wherein the system is further caused to:
identify, by a fourth machine learning model, one or more current procedural technology (CPT) codes in the electronic health data associated with the patient,
wherein the fourth machine learning model is trained to identify the one or more CPT codes from training data comprising de-identified health records paired with corresponding CPT codes, and
generate, by the fourth machine learning model, an insurance claim based on the one or more CPT codes.

18. The system of claim 11, wherein the system is further caused to:
receive, at the user interface, a user selection of a visual affordance for generating an integrated care plan;
in response to receiving the user selection of the visual affordance, generate, using the second machine learning model, an integrated care plan for the patient,
wherein the integrated care plan comprises the ordered list of the one or more recommended care providers and a natural language explanation for why each of the one or more recommended care providers were recommended; and
display the integrated care plan on the user interface.

19. The system of claim 11, wherein the system is further caused to, prior to receiving electronic health data associated with a patient:
receive unstructured electronic health information associated with a patient; and
convert, using a fast healthcare interoperability resources application programming interface (FHIR API), the unstructured electronic health information into electronic health data associated with the patient, the electronic health data comprising a FHIR data format.

20. A non-transitory computer readable storage medium storing one or more programs, the one or more programs comprising instructions, which, when executed by a system comprising one or more processors and memory, cause the system to:
receive electronic health data associated with a patient;
identify, by a first machine learning model, one or more medical condition keywords from the electronic health data associated with the patient,
wherein the first machine learning model is trained to identify medical condition keywords from training data comprising de-identified electronic health records paired with medical condition training keywords, the de-identified electronic health records comprising international classification of diseases (ICD) codes, clinical reports records, vital signs, medications, laboratory measurements, observations and notes charted by care providers, fluid balances, procedure codes, diagnostic codes, imaging reports, hospital length of stay, survival data, or any combination thereof, associated with a plurality of de-identified patients;
determine, by a second machine learning model, one or more recommended care providers for the patient based on the identified one or more medical condition keywords;
generate, using the second machine learning model, an ordered list of the one or more recommended care providers,
wherein the second machine learning model is trained to generate the ordered list from training data comprising medical condition training keywords paired with recommendations by care providers of other care providers treating one or more of the medical conditions indicated by the training keywords; and
display the ordered list of the one or more recommended care providers on a user interface.
